(19) United States
(12) Patent Application Publication
(10) Pub. No.: US 2015/0036323 A1
(43) Pub. Date: Feb. 5, 2015

(54) GOGGLES WITH LIGHTING TO PRODUCE PATTERNS OF ILLUMINATION

(71) Applicants: Joshua C. Kelly, Sellersburg, IN (US); Ariel Macaulay Bruton, Jeffersonville, IN (US); Christopher Kelly, Sellersburg, IN (US)

(72) Inventors: Joshua C. Kelly, Sellersburg, IN (US); Ariel Macaulay Bruton, Jeffersonville, IN (US); Christopher Kelly, Sellersburg, IN (US)

(21) Appl. No.: 14/332,697

(22) Filed: Jul. 16, 2014

Related U.S. Application Data
(60) Provisional application No. 61/846,622, filed on Jul. 16, 2013.
(60) Provisional application No. 61/899,239, filed on Nov. 3, 2013.

(51) Int. Cl. A61F 9/02; F21V 23/00; F21V 21/084; F21V 33/00; F21V 31/00; F21K 99/00; F21V 23/04

(52) U.S. Cl. CPC A61F 9/029; F21K 9/30; F21V 23/005; F21V 23/0414; F21V 23/0464; F21V 33/0056; F21V 31/00; F21V 21/084; F21Y 2101/02
USPC 362/103

(57) ABSTRACT

A pair of goggles that includes lighting, which produce patterns of illumination across the surface of the goggles that contains said lighting.

### CLAIM OF PRIORITY

This application claims priority to U.S. Provisional Utility Patent No. 61/846,622 filed on Jul. 16, 2013 and U.S. Provisional Patent No. 61/899,239 filed on Nov. 3, 2013. Both U.S. Provisional Utility Patent No. 61/846,622 and U.S. Provisional Patent No. 61/899,239 are incorporated herein by reference for all purposes.

### FIELD OF INVENTION

The present invention is in the field of goggles. More particularly, the present invention is related to goggles that allow for the creation of illumination patterns of light while being worn.

### BACKGROUND OF INVENTION

Goggles and sunglasses are part of normal apparel. Sunglasses use tinted surfaces to attenuate the intensity of sunlight as well as filter the light to reduce the damage of harmful UV rays on the eyes. Besides being functional, sunglasses make a style statement about the wearer. Ski goggles protect the wearer from fast moving cold air against the face and eyes, and special coatings and tinted surfaces can enhance the clarity of features being seen and make a style statement about the wearer.

The present invention is a pair of goggles covered with LED lights on the surface, which produce illumination patterns for external viewing. Many illumination patterns can be generated, including but not limited to controlled, customized or random patterns of messages, images, and pictures. Such patterns can be controlled or switched through from within the goggles or from an source that is external to the goggles. Both the illumination patterns and ability to control said patterns enhance the style statement of the wearer as well as the fun factor for the wearer and those viewing the wearer wearing the goggles.

### SUMMARY OF INVENTION

The present invention is a pair of light up goggles that permits the wearer to see through the goggles, and there can be at least one LED, and more particularly, there can be an array of LEDs covering the face of the goggles, and this array can display a variety of light patterns, messages, images, and/or pictures. The goggles can have a transparent plastic material substrate as the surface through which the wearer can see the outside world, and this substrate can serve as the mechanical support upon which at least one LED, and more particularly an LED array can be mounted. The goggles can have at least one battery, at least one switch and/or at least one sensor, and electronics to control at least one LED light, and this electronics can also control an array of LEDs to create at least one pattern. The pair of goggles can further comprise a means of distributing power to the LEDs that does not interfere with wearer's ability to see the environment.

The goggles can have at least one switch and/or at least one sensor, and these can be sensed by and/or connected to electronics. At least one sensor can be light and/or ambient light level activated, and this can control the lighting of the array of LEDs in at least one lighting sequence. Further, the switch and/or sensor can initiate and/or change at least one lighting sequence. The lighting sequence can be repetitive, random, and can be customized. The lighting can incorporate a dimming function within one or more lighting sequences. The lighting can display messages containing letters, words, numbers, symbols, equations, pictures, caricatures, and/or images. The ambient light level can determine the said at least one light sequence and/or pattern and/or intensity. At least one of the sensors can also sense acoustic energy and/or audio from the environment and convert said acoustic energy and/or audio into a signal, and this signal can be resolved into frequency and/or amplitude and/or a combination thereof, and this frequency and/or amplitude and/or a combination thereof can control or affect the pattern and/or intensity of LED lighting.

Further, there can be at least one connector to send or receive signals, and this connector and/or jack can connect to an audio source and/or power. At least one connector can also be a connector and/or jack for connecting a mobile device and/or smart device and/or computer. Signals from these devices can cause the creation of custom color and/or pattern identification of calls, e-mails, texts, and/or other origin-specific electronic communications. The goggles can also contain a communication device that can transmit and/or receive data and/or signals in a master-slave fashion via radio frequency and/or infrared and/or other methods. The transparent plastic material substrate can be waterproof and/or water resistant, and can incorporate a coating to enhance visual clarity of what is seen by the wearer, and can also be tinted to attenuate the intensity of sunlight.

Although preferred embodiments of the present invention have been described it will be understood by those skilled in the art that the present invention should not be limited to the described preferred embodiments. Rather, various changes and modifications can be made within the spirit and scope of the present invention.

### BRIEF DESCRIPTION OF DRAWINGS

FIG. 1 is a frontal view of one embodiment of the goggles showing one illuminating pattern.

FIG. 2 is three frontal views of one embodiment of the goggles with each of the three frontal views showing a different illuminating pattern.

### DETAILED DESCRIPTION OF DRAWINGS

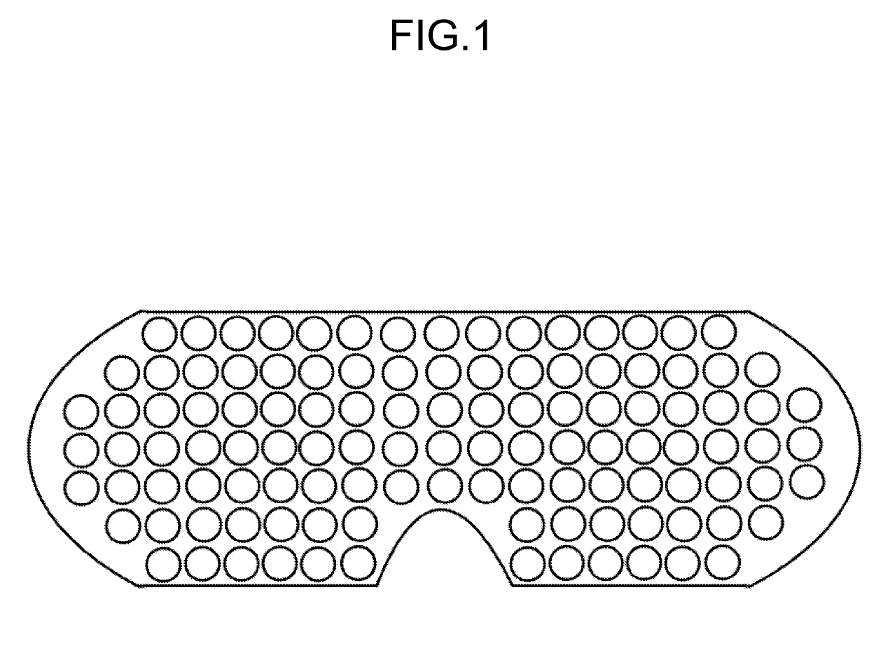

FIG. 1 is a frontal view of one embodiment of the goggles showing one illuminating pattern.

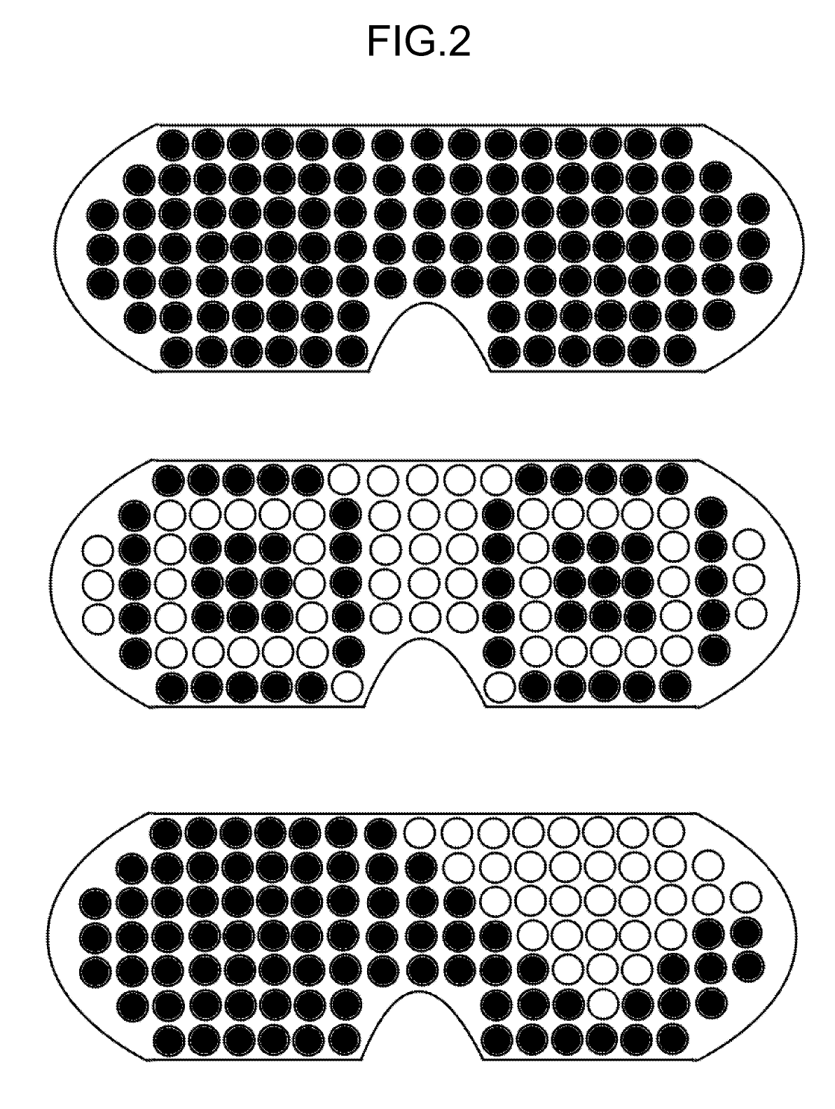

FIG. 2 is three frontal views of one embodiment of the goggles with each of the three frontal views showing a different illuminating pattern. The illuminating patterns can be internally or externally controlled.

In both FIGS. 1 and 2, the circles that are filled with the color white are non-illuminated LEDs or lights, and the circles that are filled with the color black are illuminated LEDs or light.

### DEFINITIONS

“Goggles” is defined as (1) any type of eyewear, in whole or in part, including but not limited to eyewear, eyeglasses, sunglasses, eyewear frames, shades, protective eyewear, goggles (as defined in the dictionary), ski masks, protective masks, and visors; and (2) facewear, masks, hats, belts, bracelets, jewelry, headwear, accessories, apparel, space suits, scuba and diving equipment and gear.

1. A pair of goggles comprising:
a transparent plastic material substrate;
at least one LED light;
at least one battery;
at least one switch and/or at least one sensor; and
electronics to control at least one LED light.

2. The pair of goggles in claim 1 further comprising a means of distributing power to the at least one LED light that does not interfere with the wearer's ability to see the environment.

3. The pair of goggles in claim 2 whereby said at least one LED light can be an array of LEDs covering the surface of said transparent plastic material substrate.

4. The pair of goggles in claim 3 whereby said array of LEDs are mounted to said transparent plastic material substrate for mechanical support.

5. The goggles of claim 1 whereby said at least one switch and/or at least one sensor can be sensed by and/or connected to electronics.

6. The goggles of claim 5 whereby said at least one sensor can be light and/or ambient light level activated.

7. The goggles of claim 5 whereby said electronics are able to control the illumination of said array of LEDs in at least one lighting sequence.

8. The goggles of claim 7 whereby said at least one lighting sequence can contain messages using at least one of letters, words, numbers, equations, symbols, images, pictures, caricatures, or any combination thereof.

9. The goggles of claim 8 whereby activation of said at least one switch and/or at least one sensor can initiate and/or change said at least one lighting sequence.

10. The goggles of claim 9 whereby said at least one lighting sequence can be repetitive.

11. The goggles of claim 9 whereby said at least one lighting sequence can be random.

12. The goggles of claim 9 whereby said at least one lighting sequence can be customized.

13. The goggles of claim 9 whereby said at least one lighting sequence can incorporate a dimming function.

14. The goggles of claim 6 whereby said ambient light level can determine the said at least one lighting sequence and/or pattern and/or intensity.

15. The goggles of claim 1 whereby said at least one sensor can sense acoustic energy and/or audio from the environment and convert said acoustic energy and/or audio into a signal.

16. The goggles of claim 15 whereby said signal can be resolved into frequency and/or amplitude and/or a combination thereof.

17. The goggles of claim 16 whereby said frequency and/or amplitude and/or a combination thereof can control or affect the pattern and or intensity of at least one lighting sequence.

18. The goggles of claim 1 further comprising at least one connector to send or receive signals.

19. The goggles of claim 18 whereby said at least one connector is a connector and/or jack for connecting to an audio source and/or power.

20. The goggles of claim 18 whereby said at least one connector is a connector and/or jack for connecting a mobile device and/or smart device and/or computer.

21. The goggles of claim 20 further comprising custom color and/or pattern identification of calls, e-mails, texts, and/or other origin-specific electronic communications.

22. The goggles of claim 20 further comprising a communication device that can transmit and or receive data and or signals in a master-slave fashion via radio frequency and/or infrared and/or other methods.

23. The goggles of claim 8 whereby said at least one lighting sequence can be controlled from internal settings or from external sources.

24. The goggles of claim 1 whereby said transparent plastic material substrate is waterproof and/or water resistant.

25. The goggles of claim 1 whereby said transparent plastic material substrate incorporates a coating to enhance visual clarity of what is seen by the wearer.

26. The goggles of claim 1 whereby said transparent plastic material substrate can be tinted to attenuate the intensity of sunlight.
